(19) United States
(12) Patent Application Publication
(10) Pub. No.: US 2011/0223640 A1
(43) Pub. Date: Sep. 15, 2011

(54) IMPROVED STATIN PRODUCTION

(76) Inventors: Marco Alexander Van Den Berg, Poeldijk (NL); Marcus Hans, Den Haag (NL)

(21) Appl. No.: 13/119,764

(22) PCT Filed: Sep. 21, 2009
(86) PCT No.: PCT/EP2009/062194

§ 371 Date: May 29, 2011

(30) Foreign Application Priority Data
Sep. 24, 2008 (EP) 08164979.0

(51) Int. Cl. C12P 17/06; C12N 1/15; C12N 1/14; C12P 1/02; C12P 7/62

(52) U.S. Cl. 435/125; 435/254.11; 435/254.1; 435/254.5; 435/171; 435/135

(57) ABSTRACT

The present invention provides a method for the fermentative production of compactin, lovastatin, pravastatin or simvastatin comprising culturing a host, preferably a filamentous fungus, comprising the polynucleotide of the lovE transcription regulator gene from Aspergillus terreus. Furthermore, the invention provides a host for the production of above mentioned statines comprising the polynucleotide of the lovE transcription regulator gene from Aspergillus terreus.

### FIELD OF THE INVENTION

The present invention relates to a method for fermentation of statins.

### BACKGROUND OF THE INVENTION

Cholesterol lowering agents of the statin class are important drugs as they lower the cholesterol concentration in the blood by inhibiting HMG-CoA reductase. The latter enzyme catalyses the rate limiting step in cholesterol biosynthesis, i.e. the conversion of (3S)-hydroxy-3-methylglutaryl-coenzyme A (HMG-CoA) to mevalonate. There are several types of statins on the market, amongst which atorvastatin, compactin, lovastatin, simvastatin and pravastatin. Whilst atorvastatin is made via chemical synthesis, the others mentioned are produced either via direct fermentation or via precursor fermentation. These (precursor) fermentations are carried out by fungi of the genera Penicillium, Aspergillus and Monascus.

There is a common problem while fermenting these compounds in heterologous hosts, i.e. hosts such as Penicillium chrysogenum that do not naturally produce statins and in which the complete statin pathway genes from natural statin producers (e.g. Penicillium citrinum or Aspergillus terreus) were introduced, as the productivities and yields of the heterologous strains used are low. Several technologies were published earlier and include the classical improvement of statin production strains by UV mutagenesis (J. Gen. Appl. Microbiol. (2004), Vol. 50, p. 169-176). Furthermore, a transformation of the statin production organism Penicillium citrinum with a polynucleotide corresponding to the gene mlcR from Penicillium citrinum was described. However, both technologies did not lead to improvements of statin production in heterologous hosts. Yet another approach was disclosed in WO 2007/122249 employing a Penicillium chrysogenum strain that produces on an industrial level however the problem with such an approach is that industrially producing microorganisms are not always available and/or suitable. There remains a need for alternative approaches.

### DETAILED DESCRIPTION OF THE INVENTION

The term “expression” includes any step involved in the production of a polypeptide and may include transcription, post-transcriptional modification, translation, post-translational modification and secretion.

The term “nucleic acid construct” is synonymous with the term “expression vector” or “cassette” when the nucleic acid construct contains all the control sequences required for expression of a coding sequence in a particular host organism.

The term “control sequences” is defined herein to include all components, which are necessary or advantageous for the expression of a polypeptide. Each control sequence may be native or foreign to the nucleic acid sequence encoding the polypeptide. Such control sequences may include, but are not limited to, a promoter, a leader, optimal translation initiation sequences (as described in Kozak, 1991, J. Biol. Chem. 266:19867-19870), a secretion signal sequence, a pro-peptide sequence, a polyadenylation sequence, a transcription terminator. At a minimum, the control sequences include a promoter, and transcriptional and translational stop signals. The control sequence may be an appropriate promoter sequence containing transcriptional control sequences. The promoter may be any nucleic acid sequence, which shows transcription regulatory activity in the cell including mutant, truncated, and hybrid promoters, and may be obtained from genes encoding extra cellular or intracellular polypeptides. The promoter may be either homologous or heterologous to the cell or to the polypeptide. The promoter may be derived from the donor species or from any other source. An alternative way to control expression levels in eukaryotes is the use of introns. Higher eukaryotes have genes consisting of exons and introns. The term “exons” is defined herein to include all components of the Open Reading Frame (ORF), which are translated into the protein. The term “introns” is defined herein to include all components, which are not comprised within the Open Reading Frame. The term “Open Reading Frame” is defined herein as a polynucleotide starting with the sequence ATG, the codon for methionine, followed by a consecutive series of codons encoding all possible amino acids and after a certain number interrupted by a termination codon. This Open Reading Frame can be translated into a protein. A polynucleotide containing a gene isolated from the genome is a so-called genomic DNA or gDNA sequence of that gene, including all exons and introns. A polynucleotide containing a gene isolated from mRNA via reverse transcriptase reactions is a so-called copy DNA or cDNA sequence of that gene, including only the exons, while the introns are spliced out through the cells machinery. This latter type of DNA is of particular use when expressing eukaryotic genes of interest in prokaryotic hosts. Variants of both types of DNA can also be made synthetically, which opens the possibility to either alter the exact nucleotide sequence of the introns or vary the number of introns in the gene of interest. This also opens the possibility of adding introns to genes of interest from prokaryotic origin to facilitate or improve expression in eukaryotic hosts. Also, introns can be introduced in the above named control sequences, like a promoter, a polyadenylation site or a transcription terminator. The presence, absence, variation or introduction of introns is a means of regulating gene expression levels in eukaryotes.

The term “operably linked” is defined herein as a configuration in which a control sequence is appropriately placed at a position relative to the coding sequence of the DNA sequence such that the control sequence directs the production of a polypeptide.

The term “pravastatin” is defined herein as 6′-hydroxyl substituted compactin with an α- or β-configuration at the 6′-position, or a mixture of both α- and β-configurations and includes both the closed structure (with a lactone ring) and the open structure (with a hydroxycarboxylic acid moiety).

In the context of the present invention compactin, pravastatin, wuxistatin, monacolin J, lovastatin and/or simvastatin (generally referred to as ‘statin’ or ‘statins’) ‘biosynthetic genes’ or ‘biosynthetic pathways’ include all genes encoding enzymes directly involved in the synthesis of statin molecules, all genes encoding enzymes in secretion of statin molecules and all genes encoding proteins involved in the transcriptional regulation of the genes of the first two categories. Also, included are all genes of the microbial host capable of producing statins which by over expression or inactivation cause a significant change in the production capacity (i.e. resulting in at least 20% more statin produced or in at least 20% less statin produced, respectively). Specific genes are, but not limited to: the compactin biosynthetic gene cluster of Penicillium citrinum (i.e. mlcA, mlcB, mlcC, mlcD, mlcE, mlcF, mlcH, mlcG, mlcR; see Entrez database accession number AB072893; Abe Y, Suzuki T, Ono C, Iwamoto K, Hosobuchi M and Yoshikawa H, Mol Genet Genomics 2002, 267:636-646), the lovastatin biosynthetic gene cluster of Aspergillus terreus (i.e. ORF1, ORF2, lovA, ORF5, lovC, lovD, ORF8, lovE, ORF10, lovF, ORF12, ORF13, ORF14, ORF15, ORF16, cytochrome P450 monooxygenase, ORF18; see Entrez database accession numbers AF141924 and AF141925; Kennedy J, Auclair K, Kendrew S G, Park C, Vederas J C and Hutchinson C R, Science 1999, 284:1368-1372), the monacolin K biosynthetic gene cluster of Monascus pilosus (i.e. mkA, mkB, mkC, mkD, mkE, mkF, mkG, mkH and mkI; see Entrez database accession number DQ176595, (http://www.ncbi.nlm.nih.gov/entrez/viewer.fcgi?db=nuccore&id=74275560), and substantial homologues thereof originating from other species.

The actual statin biosynthetic pathway can be obtained from a single donor host or from more than one hosts, or (partially) consists of synthetic polynucleotides.

For the purpose of the present invention, the degree of identity between two amino acid sequences refers to the percentage of amino acids that are identical between the two sequences. The degree of identity is determined using the BLAST algorithm, which is described in Altschul, et al. (J. Mol. Biol. 215: 403-410 (1990)). Software for performing BLAST analyses is publicly available through the National Center for Biotechnology Information (http://www.ncbi.nlm.nih.gov/). The BLAST algorithm parameters W, T, and X determine the sensitivity and speed of the alignment. The BLAST program uses as defaults a word length (W) of 11, the BLOSUM62 scoring matrix (see Henikoff & Henikoff, Proc. Natl. Acad. Sci. USA 89: 10915 (1989)) alignments (B) of 50, expectation (E) of 10, M=5, N=−4, and a comparison of both strands.

Substantially homologous polypeptides may contain only conservative substitutions of one or more amino acids of the specified amino acid sequences or substitutions, insertions or deletions of non-essential amino acids. Accordingly, a non-essential amino acid is a residue that can be altered in one of these sequences without substantially altering the biological function. For example, guidance concerning how to make phenotypically silent amino acid substitutions is provided in Bowie, J. U. et al. (Science 247:1306-1310 (1990)) wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selects or screens to identify sequences that maintain functionality. As the authors state, these studies have revealed that proteins are surprisingly tolerant of amino acid substitutions. The authors further indicate which changes are likely to be permissive at a certain position of the protein. For example, most buried amino acid residues require non-polar side chains, whereas few features of surface side chains are generally conserved. Other such phenotypically silent substitutions are described in Bowie et al. and the references cited therein.

The term “conservative substitution” is intended to mean that a substitution in which the amino acid residue is replaced with an amino acid residue having a similar side chain. These families are known in the art and include amino acids with basic side chains (e.g. lysine, arginine and histidine), acidic side chains (e.g. aspartic acid, glutamic acid), uncharged polar side chains (e.g., glycine, asparagines, glutamine, serine, threonine, tyrosine, cysteine), non-polar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine tryptophan, histidine).

It is an object of the present invention to provide an improved production process for statins such as compactin, lovastatin, pravastatin, simvastatin and wuxistatin, thereby overcoming the problem of low statin productivity of recombinant statin producing strains harboring transcription activators such as mlcR.

The present invention solves the problem of low statin productivity of heterologous microorganisms (i.e. microorganisms that naturally do not harbor statin biosynthetic pathways and where statin biosynthetic pathways from other organisms such as Aspergillus terreus or Penicillium citrinum were integrated). As described in the present invention, the problem is solved by addition of another transcription regulator gene of the integrated statin pathway (such as the compactin pathway from Penicillium citrinum). Preferably, the potential transcription regulator gene of the integrated statin pathway (such as the compactin pathway from Penicillium citrinum) is exchanged against a homologous activator, such as the lovastatin transcriptional activator lovE. The invention discloses that microorganisms which heterologously harbor the Penicillium citrinum statin pathway exhibit a higher statin productivity if the original transcription regulator mlcR is replaced by lovE or analogues of lovE with a high degree of homology. As lovE itself is only moderately homologous to mlcR (the identity on genetic level is 65% and the identity on amino acid level is 40%), the favorable results of the present invention are unexpectedly surprising.

In a first aspect of the present invention there is provided a fungal strain comprising a heterologous statin biosynthetic gene transcription activator.

In one embodiment provided is a statin producing strain, not being Aspergillus terreus, containing all genes necessary for statin biosynthesis and containing the lovE gene from Aspergillus terreus. Preferably there is provided a compactin producing host cell derived from a production strain, such as for instance Penicillium chrysogenum as described in WO 2007/122249. This organism underwent several rounds of classical strain improvement and subsequent process adaptations and improvements to come to the current high titer penicillin G fermentation processes. The numerous changes in the DNA of the organism resulted not only in an increased flux and yield towards the product penicillin G, but moreover also resulted in morphological changes and adaptations to the harsh conditions in 150,000-liter fermentation vessels (i.e. oxygen limitation, shear forces, glucose limitation and the like). By deleting the β-lactam biosynthetic machinery, a strain is obtained that is devoid of any β-lactam production capability, but still retains all the mutations that result in the good performance on industrial scale, such as resistance to shear forces, suitability for scaling up, high metabolic flux towards metabolites, adapted to a defined medium and to industrial down stream processing, and low viscosity profile (i.e. morphological, regulatory and metabolic mutations). In the Penicillin chrysogenum strain of the present invention, at least the β-lactam biosynthetic genes pcbC, encoding for isopenicillin N synthase, are inactivated. Preferably, also the other β-lactam biosynthetic genes, pcbAB, encoding for L-(α-aminoadipyl)-L-cysteinyl-D-valine synthetase, and/or penDE, encoding for acyl-coenzyme A:isopenicillin N acyltransferase, are inactivated. More preferably, the genes are inactivated by removal of part of the genes. Most preferred is that the gene sequences are completely removed. As complete removal of these genes leads to Penicillium chrysogenum strains that are devoid of any β-lactam biosynthetic capacity and therefore are very useful strains for producing all sorts of products. Despite the fact that industrial organisms can be very cumbersome to work with, this Penicillium chrysogenum strain is surprisingly well transformable and capable of producing statins at titers much higher than the natural producing host cells.

Although not mandatory for the present invention, preferably the Penicillium chrysogenum mutant is obtained from an organism capable of producing in an industrial environment. Such organisms typically can be defined as having high productivities and/or high yield of product on amount of carbon source consumed and/or high yield of product on amount of biomass produced and/or high rates of productivity and/or high product titers. Such organisms are extremely useful for conversion into a host cell for compactin. For penicillin G producing Penicillium chrysogenum strains for instance, such high titers are titers higher than 1.5 g/L penicillin G, preferably higher than 2 g/L penicillin G, more preferably higher than 3 g/L penicillin G, most preferably higher than 4 g/L penicillin G. The aforementioned values apply to fermentation titers after 96 h in complex fermentation medium (contains per liter: lactose, 40 g/L; corn steep solids, 20 g/L; CaCO₃, 10 g/L; KH₂PO₄, 7 g/L; phenylacetic acid, 0.5 g/L; pH 6.0). Suitable industrial strains are strains as mentioned in the experimental part (General Methods).

All industrial strain lineages of Penicillium chrysogenum underwent numerous rounds of classical strain improvement resulting in three general types of mutations:
(i) Direct amplification of the biosynthetic genes resulting in increased activity of the enzymes of the penicillin metabolite pathway
(ii) Modifications in primary metabolism genes, ultimately resulting in various adapted metabolic rearrangements, all leading to higher a higher flux towards the end product. Examples: increased synthesis of amino acid building blocks, decreased consumption of phenylacetic acid and the like.
(iii) Cell structure modifications, resulting in alteration of morphology, membrane composition, organelles organization and thereby ‘facilitating’ high metabolic fluxes and fermentation at industrial scale. Examples: increased numbers of peroxisomes, which are one of the ‘assembly lines’ of penicillin synthesis.

There is a significant distinction on DNA level in the type of mutations of class (i) as compared to classes (ii) and (iii). While the latter two classes are mostly isolated mutations, deletions, duplications and/or alterations on base pair level, the mutation in class (i) is a very distinct amplification of a 60 to 100 kb region, resulting in several direct and inverted repeats on the genome. This sometimes causes a significant genetic instability, resulting in an instable and changing population. In fact this means that in a given penicillin production strain all mutations of class (ii) and (iii) are fixed, but the exact copy number of the mutation of class (i) can fluctuate. Using this principle and techniques known to the ones trained in the art, stable isolates can be obtained where only one copy of the penicillin biosynthetic genes is still present. Depending on the copy number of the starting strain this situation can be obtained in one, two, three or several rounds of screening and selection. For this specific characteristic the isolate is then comparable to the type strain of the species, NRRL1951, and its first descendants after classical strain improvement, up to Wisconsin 54-1255, all of which contain one copy of the penicillin biosynthetic genes. The major difference is that the one-copy isolate derived from the high producing strain still contains all the other mutations of class (ii) and (iii) making it an industrial high producing strain as compared to the strains from NRRL1951 to Wisconsin 54-1255. Subsequently, the last set of penicillin biosynthetic genes can be deleted using state-of-the-art recombination techniques. A detailed overview of these steps is given in the examples and summarized in the following steps:
(a) Isolating an isolate with a single genomic copy of the penicillin gene cluster from a Penicillium strain
(b) Deleting gene pcbC from the isolate obtained in step (a)
(c) Optionally deleting genes pcbAB and/or penDE from the isolate obtained in steps (a) or (b)

The genes can be partly inactivated. More preferably, the gene sequences are completely removed. As complete removal of these genes leads to Penicillium chrysogenum strains that are devoid of any β-lactam biosynthetic capacity and therefore are very useful strains for producing all sorts of products. Recombination techniques that can be applied are well known for the ones trained in the art (i.e. Single Cross Over or Double Homologous Recombination).

A preferred strategy for deletion and replacement is the gene replacement technique described in EP 357127. The specific deletion of a gene and/or promoter sequence is preferably performed using the amdS gene as selection marker gene as described in EP 635574. By means of counter selection on fluoroacetamide media (EP 635574), the resulting strain is selection marker free and can be used for further gene modifications. Alternatively or in combination with other mentioned techniques, a technique based on in vivo recombination of cosmids in Escherichia coli can be used, as described by Chaveroche et al. (2000, Nucl Acids Res, 28, E97). This technique is applicable to other filamentous fungi like for example Penicillium chrysogenum. Also, the same principle for removing amplified genome fragments can be applied to other industrial production species in which classical strain improvement programs have induced gene and genome duplications. Also, here additional mutations of class (ii) and (iii) are fixed and make sure that the strains can thrive in industrial fermentation processes.

Such Penicillium chrysogenum cells can be equipped with the genes encoding all proteins and enzymes necessary for compactin biosynthesis. This can be one or more of the statin biosynthetic genes, for example the eight genes of Penicillium citrinum described as being involved in compactin biosynthesis (Abe et al., 2002, Mol Genet Genomics 267:636-646; Abe et al., 2002, Mol Genet Genomics 268:130-137): mlcA, encoding a polyketide synthase; mlcB, encoding a polyketide synthase; mlcC, encoding P450 monooxygenase; mlcD, encoding a HMG-CoA reductase; mlcE, encoding an efflux pump; mlcF, encoding an oxidoreductase; mlcG, encoding a dehydrogenase; mlcH, encoding transesterase. Abe et al. also described a transcription regulator mlcR which is essential for compactin biosynthesis and which can also increase compactin levels of a Penicillium citrinum strain when added to the cell. Also, any homologous gene displaying the similar activity can be used.

In the present invention it is disclosed that the transcription regulator gene mlcR can be replaced by lovE from Aspergillus terreus. The LovE transcription regulator protein shares 40% sequence identity to the transcription activator MlcR from Penicillium citrinum. It is therefore not to be expected that lovE can take over the function of mlcR. Surprisingly, however the LovE transcription regulator protein not only can take over the function of the transcription activator MlcR from Penicillium citrinum, it even results in improved productivity. Alternatively, a gene homologous to the lovE gene can be used to improve the statin production in the heterologous host cell. Preferably the gene should be 70% homologous to the lovE gene; more preferably the gene should be 80% homologous to the lovE gene; even more preferably the gene should be 90% homologous to the lovE gene. The same principle can be applied to come to a suitable compactin producing host cell of other eukaryotic species, that are not Aspergillus terreus, and their industrial derivatives, like, but not limited to: Aspergillus niger, Penicillium brevicompactum, Penicillium citrinum, Aspergillus oryzae, Trichoderma reesei, Chrysosporium lucknowense, Saccharomyces cerevisiae, Kluyveromyces lactis, Monascus ruber, Monascus paxii, Mucor hiemalis, Pichia ciferrii and Pichia pastoris. The industrial derivatives of these species underwent various rounds of classical mutagenesis, followed by screening and selection for improved industrial production characteristics, which make them of particular use for the present invention. By removing (i.e. deleting) parts of or complete pathways of unwanted products the strains remain their desired industrial fermentation characteristics and high flux to metabolites (including enzymes).

The production of compactin can be improved by using homologous proteins with improved kinetic features. Such a “homologue” or “homologous sequence” is defined as a DNA sequence encoding a polypeptide that displays at least one activity of the polypeptide encoded by the original DNA sequence isolated from the donor species and has an amino acid sequence possessing a degree of identity to the amino acid sequence of the protein encoded by the specified DNA sequence. A polypeptide having an amino acid sequence that is “substantially homologous” to the compactin biosynthetic genes are defined as polypeptides having an amino acid sequence possessing a degree of identity to the specified amino acid sequence of at least 25%, more preferably at least 30%, more preferably at least 40%, more preferably at least 50%, still more preferably at least 60%, still more preferably at least 70%, still more preferably at least 80%, still more preferably at least 90%, still more preferably at least 98% and most preferably at least 99%, the substantially homologous peptide displaying activity towards the synthesis of compactin and/or compactin-precursors. Using this approach various advantages can be obtained such as to overcome feedback inhibition, improvement of secretion and reduction of byproduct formation. A homologous sequence may encompass polymorphisms that may exist in cells from different populations or within a population due to natural allelic or intra-strain variation. A homologue may further be derived from a species other than the species where the specified DNA sequence originates from, or may be artificially designed and synthesized. DNA sequences related to the specified DNA sequences and obtained by degeneration of the genetic code are also part of the invention. Homologues may also encompass biologically active fragments of the full-length sequence.

Of particular interest are homologous sequences isolated by synthetic means. By this method all possible variants of the genes to be introduced in the compactin producing host cell can be designed in silico. This opens the opportunity to adapt the codon usage of the genes such that they are optimally expressed in the compactin producing host cell; remove and introduce relevant sequences for restriction enzymes and/or site-specific recombinases and make different combinations of genes.

Additionally, biosynthetic gene clusters that are not homologous, but follow the same biosynthetic building principle for statin synthesis can be used.

The nucleic acid constructs of the present invention, e.g. expression constructs, contain at least one gene of interest, but in general contain several genes of interest; each operably linked to one or more control sequences, which direct the expression of the encoded polypeptide in the compactin producing host cell. The nucleic acid constructs may be on one DNA fragment or on separate fragments. To obtain the highest possible productivity a balanced expression of all genes of interests is crucial. Therefore, a range of promoters can be useful. Preferred promoters for application filamentous fungal cells like Penicillium chrysogenum are known in the art and can be, for example, the promoters of the gene(s) derived Penicillium citrinum; the glucose-6-phosphate dehydrogenase gpdA promoters; the Penicillium chrysogenum pcbAB, pcbC and penDE promoters; protease promoters such as pepA, pepB, pepC; the glucoamylase glaA promoters; amylase amyA, amyB promoters; the catalase catR or catA promoters; the glucose oxidase goxC promoter; the beta-galactosidase lacA promoter; the α-glucosidase aglA promoter; the translation elongation factor tefA promoter; xylanase promoters such as xlnA, xlnB, xlnC, xlnD; cellulase promoters such as eglA, eglB, cbhA; promoters of transcriptional regulators such as areA, creA, xlnR, pacC, prtT, alcR, or any other. Said promoters can easily be found by the skilled person, amongst others, at the NCBI Internet website (http://www.ncbi.nlm.nih.gov/entrez/). In case of compactin producing host cells derived from other than filamentous fungal species the choice of promoters will be determined by the choice of the host.

Preferably, the promoters are derived from genes that are highly expressed (defined herein as the mRNA concentration with at least 0.5% (w/w) of the total cellular mRNA). The promoters may be derived from genes, which are medium expressed (defined herein as the mRNA concentration with at least 0.01% until 0.5% (w/w) of the total cellular mRNA). In another preferred embodiment, the promoters may be derived from genes, which are low expressed (defined herein as the mRNA concentration lower than 0.01% (w/w) of the total cellular mRNA). More preferably, micro array data is used to select genes, and thus promoters of those genes, that have a certain transcriptional level and regulation. In this way one can adapt the gene expression cassettes optimally to the conditions it should function in. These promoter fragments can be derived from many sources, i.e. different species, PCR amplified, synthetically and the like.

The control sequence may also include a suitable transcription termination sequence, a sequence recognized by a eukaryotic cell to terminate transcription. The terminator sequence is operably linked to the 3′-terminus of the nucleic acid sequence encoding the polypeptide. Any terminator, which is functional in the cell, may be used in the present invention. Preferred terminators for filamentous fungal cells are obtained from the genes encoding Aspergillus oryzae TAKA amylase; the Penicillium chrysogenum pcbAB, pcbC and penDE terminators; Aspergillus niger glucoamylase; Aspergillus nidulans anthranilate synthase; Aspergillus niger alpha-glucosidase; Aspergillus nidulans trpC gene; Aspergillus nidulans amdS; Aspergillus nidulans gpdA; Fusarium oxysporum trypsin-like protease. Even more preferred terminators are the ones of the gene(s) derived from the natural producer, Penicillium citrinum. In case of compactin producing host cells derived from other than filamentous fungal species the choice of termination sequences will be determined by the choice of the host.

The control sequence may also be a suitable leader sequence, a non-translated region of an mRNA that is important for translation by the cell. The leader sequence is operably linked to the 5′-terminus of the nucleic acid sequence encoding the polypeptide. Any leader sequence, which is functional in the cell, may be used in the present invention. Preferred leaders for filamentous fungal cells are obtained from the genes encoding Aspergillus oryzae TAKA amylase and Aspergillus nidulans triose phosphate isomerase and Aspergillus niger glaA.

The control sequence may also be a polyadenylation sequence, which is operably linked to the 3′-terminus of the nucleic acid sequence and which, when transcribed, is recognized by the filamentous fungal cell as signal to add polyadenosine residues to transcribed mRNA. Any polyadenylation sequence, which is functional in the cell, may be used in the present invention. Preferred polyadenylation sequences for filamentous fungal cells are obtained from the genes encoding Aspergillus oryzae TAKA amylase, Aspergillus niger glucoamylase, Aspergillus nidulans anthranilate synthase, Fusarium oxysporum trypsin-like protease and Aspergillus niger alpha-glucosidase.

For a polypeptide to be secreted, the control sequence may also include a signal peptide-encoding region, coding for an amino acid sequence linked to the amino terminus of the polypeptide, which can direct the encoded polypeptide into the cell's secretory pathway. The 5′-end of the nucleic acid coding sequence may inherently contain a signal peptide-coding region naturally linked in translation reading frame with the segment of the coding region, which encodes the secreted polypeptide. Alternatively, the 5′-end of the coding sequence may contain a signal peptide-coding region, which is foreign to the coding sequence. The foreign signal peptide-coding region may be required where the coding sequence does not normally contain a signal peptide-coding region. Alternatively, the foreign signal peptide-coding region may simply replace the natural signal peptide-coding region in order to obtain enhanced secretion of the polypeptide.

In case of eukaryotic compactin producing host cells the control sequence may include organelle targeting signals. Such a sequence is encoded by an amino acid sequence linked to the polypeptide, which can direct the final destination (i.e. compartment or organelle) within the cell. The 5′- or 3′-end of the coding sequence of the nucleic acid sequence may inherently contain these targeting signals coding region naturally linked in translation reading frame with the segment of the coding region, which encodes the polypeptide. The various sequences are well known to the persons trained in the art and can be used to target proteins to compartments like mitochondria, peroxisomes, endoplasmatic reticulum, golgi apparatus, vacuole, nucleus and the like.

The nucleic acid construct may be an expression vector. The expression vector may be any vector (e.g. a plasmid or virus), which can be conveniently subjected to recombinant DNA procedures and can bring about the expression of the nucleic acid sequence encoding the polypeptide. The choice of the vector will typically depend on the compatibility of the vector with the cell into which the vector is to be introduced. The vectors may be linear or closed circular plasmids. The vector may be an autonomously replicating vector, i.e. a vector, which exists as an extra chromosomal entity, the replication of which is independent of chromosomal replication, e.g. a plasmid, an extra chromosomal element, a mini chromosome, or an artificial chromosome. An autonomously maintained cloning vector for a filamentous fungus may comprise the AMA1-sequence (see e.g. Aleksenko and Clutterbuck (1997), Fungal Genet. Biol. 21: 373-397). Alternatively, the vector may be one which, when introduced into the cell, is integrated into the genome and replicated together with the chromosome(s) into which it has been integrated. The integrative cloning vector may integrate at random or at a predetermined target locus in the chromosomes of the host cell. In a preferred embodiment of the invention, the integrative cloning vector comprises a DNA fragment, which is homologous to a DNA sequence in a predetermined target locus in the genome of host cell for targeting the integration of the cloning vector to this predetermined locus. In order to promote targeted integration, the cloning vector is preferably linearized prior to transformation of the host cell. Linearization is preferably performed such that at least one but preferably either end of the cloning vector is flanked by sequences homologous to the target locus. The length of the homologous sequences flanking the target locus is preferably at least at least 0.1 kb, even preferably at least 0.2 kb, more preferably at least 0.5 kb, even more preferably at least 1 kb, most preferably at least 2 kb.

The efficiency of targeted integration of a nucleic acid construct into the genome of the host cell by homologous recombination, i.e. integration in a predetermined target locus, is preferably increased by augmented homologous recombination abilities of the host cell. Such phenotype of the cell preferably involves a deficient hdfA or hdfB gene as described in WO 05/95624, and any improvements of this. WO 05/95624 discloses a preferred method to obtain a filamentous fungal cell comprising increased efficiency of targeted integration.

The vector system may be a single vector or plasmid or two or more vectors or plasmids, which together contain the total DNA to be introduced into the genome of the host cell. However in the present invention the constructs are preferably integrated in the genome of the host strain. As this is a random process this even can result in integration in genomic loci, which are highly suitable to drive gene expression, resulting in high amounts of enzyme and subsequently in high productivity.

Fungal cells may be transformed by protoplast formation, protoplast transformation, and regeneration of the cell wall. Suitable procedures for transformation of fungal host cells are described in EP 238023 and Yelton et al. (1984, Proc. Natl. Acad. Sci. USA 81:1470-1474). Suitable procedures for transformation of filamentous fungal host cells using Agrobacterium tumefaciens are described by de Groot M. J. et al. (1998, Nat Biotechnol 16:839-842; Erratum in: 1998, Nat Biotechnol 16:1074). Other methods like electroporation, described for Neurospora crassa, may also be applied.

Fungal cells are transformed using co-transformation, i.e. along with gene(s) of interest also a selectable marker gene is transformed. This can be either physically linked to the gene of interest (i.e. on a plasmid) or on a separate fragment. Following transfection transformants are screened for the presence of this selection marker gene and subsequently analyzed for the presence of the gene(s) of interest. A selectable marker is a product, which provides resistance against a biocide or virus, resistance to heavy metals, prototrophy to auxotrophs and the like. Useful selectable markers include amdS (acetamidase), argB (ornithinecarbamoyltransferase), bar (phosphinothricinacetyltransferase), hygB (hygromycin phosphotransferase), niaD (nitrate reductase), pyrG (orotidine-5′-phosphate decarboxylase), sC or sutB (sulfate adenyltransferase), trpC (anthranilate synthase), ble (phleomycin resistance protein), or equivalents thereof.

The same principle can be applied to come to a suitable compactin producing host cell of prokaryotic species, and their industrial derivatives, like, but not limited to: Streptomyces clavuligerus, Streptomyces avermitilis, Streptomyces peucetius, Streptomyces coelicolor, Streptomyces lividans, Streptomyces carbophilus, Amycolatopsis orientalis, Corynebacterium glutamicum and Escherichia coli. The industrial derivatives of these species underwent various rounds of classical mutagenesis, followed by screening and selection for improved industrial production characteristics, which make them of particular use for the present invention. By removing (i.e. deleting) parts of or complete pathways of unwanted products the strains remain their desired industrial fermentation characteristics and high flux to metabolites (including enzymes). Here, all the statin biosynthetic genes need be modified by state-of-the art methods to be functionally expressed in prokaryotic cells. The ones trained in the art will understand that this involves various steps comparable as outlined above for eukaryotes, including, but not limited to:
obtaining cDNA or synthetic DNA (to exclude eukaryotic introns)
optionally using codon-optimization
equipping with promoters functional in prokaryotes
equipping with terminators functional in prokaryotes
introducing via vectors functional in prokaryotes

In a second embodiment of the invention there is provided a host microorganism comprising the genes necessary for converting compactin into pravastatin. Preferably a fungal host is used comprising the genes necessary for compactin biosynthesis (one or more of the following genes: mlcA, encoding a polyketide synthase; mlcB, encoding a polyketide synthase; mlcC, encoding P450 monooxygenase; mlcD, encoding a HMG-CoA reductase; mlcE, encoding an efflux pump; mlcF, encoding an oxidoreductase; mlcG, encoding a dehydrogenase; mlcH, encoding transesterase, lovE, transcription regulator, including all homologous functionally active genes) and additionally integrating a gene encoding for a P450 compactin hydroxylase. Preferably, this P450 compactin hydroxylase gene is from Amycolatopsis orientalis. More preferably, the P450 compactin hydroxylase gene is 80% homologous to SEQ ID NO 3 or SEQ ID NO 6 from WO 2008/071673. Even more preferably, the P450 compactin hydroxylase gene is identical to SEQ ID NO 3 or SEQ ID NO 5 from WO 2008/071673. The scope of the invention is not limited to these specific examples.

In a third embodiment there is disclosed a host microorganism providing the genes necessary for the production of the statin monacolin J. A fungal host is used which is not Aspergillus terreus. Most preferably the fungal host is Penicillium chrysogenum. The fungal host comprises the genes needed for monacolin J biosynthesis (all mlc genes are described in Abe et al., 2002, Mol Genet Genomics 267:636-646; Abe et al., 2002, Mol Genet Genomics 268:130-137, while the lov genes are described in Kennedy et al., 1999, Science 284, 1368-1372): lovB, encoding a polyketide synthase; mlcC, encoding P450 monooxygenase; mlcD, encoding a HMG-CoA reductase; mlcE, encoding an efflux pump; mlcF, encoding an oxidoreductase; mlcG, encoding a dehydrogenase; mlcH, encoding transesterase, lovE, transcription regulator. The production of monacolin J by employing such a microbial host can be further improved by using homologous proteins with improved kinetic features, as described in the present invention.

In a fourth embodiment a microorganism is disclosed providing the genes needed for the production of lovastatin. The microorganism, which is preferably a fungus, but not Aspergillus terreus, even more preferably a filamentous fungus, most preferably Penicillium chrysogenum, can comprise one or more of the following set of genes leading to the production of lovastatin: (all mlc genes are described in Abe et al., 2002, Mol Genet Genomics 267:636-646; Abe et al., 2002, Mol Genet Genomics 268:130-137, while the lov genes are described in Kennedy et al., 1999, Science 284, 1368-1372): mlcB, encoding a polyketide synthase, lovB, encoding a polyketide synthase; mlcC, encoding P450 monooxygenase; mlcD, encoding a HMG-CoA reductase; mlcE, encoding an efflux pump; mlcF, encoding an oxidoreductase; mlcG, encoding a dehydrogenase; mlcH, encoding transesterase, lovE, transcription regulator. The invention is not restricted to the mentioned genes, but does also include homologous proteins.

In a fifth embodiment of the present invention, a monacolin J producing microorganism is disclosed that can be furthermore used for the production of simvastatin. During the growth of the monacolin J producing microorganism, the compound 2,2-dimethylbutyrate or a 2,2-dimethylbutyrate precursor such as 2,2-dimethylbutyrate ester, most preferably 2,2-dimethylbutyrate thioester is added to the culture. As a consequence, simvastatin is being produced. In order to form the thioester compounds of 2,2-dimethylbutyrate, preferably the thiol compounds methylmercaptopropionate, ethylmercaptopropionate or N-acetylcysteamin are employed. The invention is however not restricted to the use of the disclosed thioesters. Other thioesters are also suitable compounds for the invention. Alternatively, the production organism can be modified in such a way that it produces the 2,2-dimethylbutyrate side chain it self from the raw materials supplied.

In a second aspect of the present invention there is disclosed a method for producing a statin in a fungal strain as described in the first aspect of the invention.

In a first embodiment said statin is pravastatin and the method of the present invention preferably comprises the steps of:
(a) Supplying a host cell with one or more polynucleotide comprising the genes of interest encoding the minimal requirements for compactin biosynthesis;
(b) Transforming said host cell with a polynucleotide comprising the gene of interest encoding compactin hydroxylase and/or with a polynucleotide comprising a DNA sequence affecting the expression of the gene of interest;
(c) Selecting clones of transformed cells;
(d) Cultivating said selected cells;
(e) Feeding nutrient sources to said cultivated cells, and;
(f) Optionally isolating pravastatin from said cultivations.

In a second embodiment the production of pravastatin in a compactin hydroxylase expressing host cell can be improved by using homologous proteins with improved kinetic features. Such a “homologue” or “homologous sequence” is defined as a DNA sequence encoding a polypeptide that displays at least one activity of the polypeptide encoded by the original DNA sequence isolated from the donor species and has an amino acid sequence possessing a degree of identity to the amino acid sequence of the protein encoded by the specified DNA sequence. A polypeptide having an amino acid sequence that is “substantially homologous” to the compactin hydroxylase genes is defined as a polypeptide having an amino acid sequence possessing a degree of identity to the specified amino acid sequence of at least 25%, more preferably at least 30%, more preferably at least 40%, more preferably at least 50%, still more preferably at least 60%, still more preferably at least 70%, still more preferably at least 80%, still more preferably at least 90%, still more preferably at least 98% and most preferably at least 99%, the substantially homologous peptide displaying activity towards the synthesis of pravastatin. Using this approach various advantages are obtained such as to overcome feedback inhibition, improvement of secretion and reduction of byproduct formation. A homologous sequence may encompass polymorphisms that may exist in cells from different populations or within a population due to natural allelic or intra-strain variation. A homologue may further be derived from a species other than the species where the specified DNA sequence originates from, or may be artificially designed and synthesized. DNA sequences related to the specified DNA sequences and obtained by degeneration of the genetic code are also part of the invention. Particularly important are homologous sequences isolated synthetically. By this method all possible variants of the genes encoding suitable p450 enzymes can be designed in silico. This opens the opportunity to adapt the codon usage of the genes in such a way that they are optimally expressed in either the compactin producing host cell and/or the compactin hydroxylase expressing host cell; remove and introduce relevant sequences for restriction enzymes and/or site-specific recombinases, make different combinations of the genes, etc. Alternatively, one can use in vitro evolutionary approaches like error prone PCR, family shuffling and/or directed evolution as methods to obtain homologous sequences with improved kinetic properties. Homologues may also encompass biologically active fragments of the full-length sequence. Additionally, genes that are not homologous, but do catalyze the formation of pravastatin from compactin or any of the compactin-precursors can be used.

In a third embodiment the efficiency of the compactin to pravastatin conversion may be improved by isolating a specific redox regenerating system, needed for the p450 enzyme, and introducing this in the compactin hydroxylase expressing host cell. The methods of introducing such a system in the host cell are the same as described for introducing the compactin hydroxylase as outlined above. Such a redox regenerating system may be obtained from the same species as from which the polynucleotide encoding the compactin hydroxylase (i.e. p450) may be obtained or heterologously expressed in; examples of which are Penicillium species (i.e. Penicillium chrysogenum, Penicillium citrinum), Aspergillus species (i.e. Aspergillus niger, Aspergillus nidulans, Aspergillus terreus), Mucor species (i.e. Mucor hiemalis), Monascus species (i.e. Monascus ruber, Monascus paxii), Streptomyces species (i.e. Streptomyces carbophilus, Streptomyces flavidovirens, Streptomyces coelicolor, Streptomyces lividans, Streptomyces exfoliates, Streptomyces avermitilis, Streptomyces clavuligerus), Amycolatopsis species (i.e. Amycolatopsis orientalis NRRL 18098, Amycolatopsis orientalis ATCC 19795), Bacillus species (i.e. Bacillus subtilus, Bacillus amyloliquefaciens, Bacillus licheniformis), Corynebacterium species (i.e. Corynebacterium glutamicum), or Escherichia species (i.e. Escherichia coli). Also, alternative systems can be applied. Examples of alternative systems are, but are not limited to, integrating the compactin hydroxylase of the present invention in a class IV p450 system, thereby fusing it to the redox partners (see for example Roberts et al., 2002, J Bacteriol 184:3898-3908 and Nodate et al., 2005, Appl Microbiol Biotechnol Sep 30:1-8) or by NAD(P)H generating non-p450 linked enzymes like phosphite dehydrogenase (Johannes et al., 2005, Appl Environ Microbiol. 71:5728-5734.) or by non-enzymatic means (see for example Hollmann et al., 2006, Trends Biotechnol 24:163-171). The host cell thus obtained may be used for producing pravastatin.

In a fourth embodiment the one-step fermentation of pravastatin is carried out by mixing a compactin producing host cell with a compactin hydroxylase expressing host cell, and subsequently cultivating both host cells as a mixed culture, understanding that the compactin produced and secreted by the compactin producing host cell, will be imported and converted to pravastatin by the compactin hydroxylase expressing host cell.

In a fifth embodiment of the present invention, the same principle to improve statin productivity can be applied to Aspergillus terreus strains producing monacolin J and/or lovastatin: increase the transcription of the biosynthetic genes by addition of exchange of a heterologous transcription activator, in this case MlcR, or any homologous gene or protein.

In a third aspect of the invention, the use of a strain of the first aspect is disclosed. The microorganism of the first aspect is ideally suitable for the production of statins, such as compactin, pravastatin, lovastatin, simvastatin and wuxistatin. The scope of the invention is not limited to these mentioned examples.

### EXAMPLES

### General Methods

Standard DNA procedures were carried out as described elsewhere (Sambrook et al., 1989, Molecular cloning: a laboratory manual, 2^{nd} Ed., Cold Spring Harbor Laboratory Press, Cold Spring Harbor, N.Y.). If specific DNA methods were applied these are specified. DNA was amplified using the proofreading enzyme like Phusion polymerase (Finnzyme) or Herculase polymerase (Stratagene). Restriction enzymes were from Invitrogen or New England Biolabs. All Penicillium chrysogenum strains used were described in the patent application WO 2007/147827. The construction of the strain referred to as “beta-lactam minus” strain in the following examples is described in comparative Example 3 of WO 2007/147827.

Fungal growth was performed in a mineral medium, containing (g/L): glucose (5); lactose (35); urea (4.5); (NH₄)₂SO₄ (1.1); Na₂SO₄ (2.9); KH₂PO₄ (5.2); K₂HPO₄ (4.8) and 10 mL/L of a trace element solution containing (in g/l): citric acid (150); FeSO₄.7H₂O (15); MgSO₄.7H₂O (150); H₃BO₃ (0.0075); CuSO₄.5H₂O (0.24); CoSO₄.7H₂O (0.375); ZnSO₄.7H₂O (5); MnSO₄.H₂O (2.28); CaCl₂.2H₂O (0.99); pH before sterilization 6.5.

### Example 1

### Isolation of the Compactin Gene Cluster from Penicillium citrinum NRRL8082

Chromosomal DNA was isolated from Penicillium citrinum NRRL8082. As the full gene cluster is difficult to amplify via PCR due to its size, it was divided in three fragments: 18 kb, 14 kb and 6 kb. The 14 and 6 kb fragments, were readily PCR amplified and cloned using Gateway (Invitrogen) into the entry vectors pDONR221 and pDONRP2-P3 with a so-called LR gateway reaction according to the suppliers' instructions. The 18 kb fragment was cloned in a two-step procedure. First, a 10 and an 8 kb fragment were amplified. Both fragments were cloned separately in pCR2.1 TOPO T/A (Invitrogen) and subsequently fused together via restriction enzyme cloning and ligation as described in WO 2007/147827. Finally, the fragment was transferred to the pDONR41Zeo vector using Gateway technology. The amplified fragments were verified via sequencing. Using a so-called Multi-site Gateway Reaction (see manual Invitrogen) these three gene fragments containing all the genes of the compactin biosynthetic gene clusters can be recombined into one fragment, spanning the whole region.

TABLE 1
Oligonucleotides used to amplify the compactin biosynthetic gene cluster

| | Forward primer | | Reverse primer | |
|---|---|---|---|---|
| | Cluster | Gateway | Cluster | Gateway |
| Target DNA | sequence | Sequence | sequence | Sequence |
| Left part of the | SEQ ID NO 1 | attB4 | SEQ ID NO 2 | — |
| compactin cluster | | | | |
| (10 kb fragment) | | | | |
| Left part of the | SEQ ID NO 3 | — | SEQ ID NO 4 | attB1 |
| compactin cluster | | | | |
| (8 kb fragment) | | | | |
| Internal 6 kb of | SEQ ID NO 5 | — | SEQ ID NO 6 | — |
| left part of | | | | |
| compactin cluster | | | | |
| Middle part of the | SEQ ID NO 7 | attB1 | SEQ ID NO 8 | attB2 |
| compactin cluster | | | | |
| (14 kb fragment) | | | | |
| Right part of the | SEQ ID NO 9 | attB2 | SEQ ID NO 10 | attB3 |
| compactin cluster | | | | |
| (6 kb fragment) | | | | |

### Example 2

### Transformation of Penicillium chrysogenum with the Three Compactin Gene Cluster Fragments from Penicillium citrinum

The three compactin gene cluster fragments were co-transformed to the Penicillium chrysogenum β-lactam minus strain (as described in Example 3 in WO2007/147827) with a ble expression cassette encoding for a protein that mediates phleomycin resistance. This cassette can be isolated as a 1.4 kb SalI fragment from pAMPF7 (Fierro et al., 1996, Curr. Genet. 29:482-489). Selection of transformants was done on mineral medium agar plates with 50 μg/mL Phleomycin and 1 M saccharose. Phleomycin resistant colonies appearing on these protoplast regeneration plates were re-streaked on fresh phleomycin agar plates (100 μg/mL) without the saccharose and grown until sporulation. The phleomycin resistant transformants were screened via colony PCR for the presence of one or more compactin gene fragments. For this, a small piece of colony material was suspended in 50 μl TE buffer (Sambrook et al., 1989) and incubated for 10 min at 95° C. To discard the cell debris the mixture was centrifuged for 5 minutes at 3000 rpm. The supernatant (5 μl) was used as a template for the PCR-reaction with SUPER TAQ from HT Biotechnology Ltd. The PCR-reactions were analyzed on the E-gel96 system from Invitrogen.

TABLE 2
Oligonucleotides used in colony PCR for determining the
presence of the compactin biosynthetic gene cluster

| Target DNA | Forward primer | Reverse primer |
|---|---|---|
| 18 kb fragment | SEQ ID NO 11 | SEQ ID NO 12 |
| 14 kb fragment | SEQ ID NO 13 | SEQ ID NO 14 |
| 6 kb fragment | SEQ ID NO 15 | SEQ ID NO 16 |
| niaA | SEQ ID NO 17 | SEQ ID NO 18 |

First, the presence of the 18 kb fragment was checked. Out of 480 colonies checked 112 had the 18 kb fragment stably integrated (˜23%). Subsequently, the presence of the other two fragments (14 and 6 kb) was verified. Forty-five of the 18 kb-positive transformants also had both other parts of the compactin gene cluster and thereby qualified as putative compactin production strains.

Shake Flask Analysis of the Penicillium chrysogenum Transformants.

The Penicillium chrysogenum platform strain transformants with the full compactin gene cluster were evaluated in liquid mineral media as described under General Methods for the presence of (hydrolyzed) compactin (ML236B) and deacylated compactin ML-236A (6-(2-(1,2,6,7,8,8a-hexahydro-8-hydroxy-2-methyl-1-naphthalenyl)ethyl)-tetrahydro-4-hydroxy-2H-pyran-2-one). After 168 h of cultivation at 25° C. in 25 ml the supernatant was analyzed with HPLC using the following equipment and conditions:
Column: Waters XTerra RP18
Column Temp: Room temperature
Flow: 1 ml/min
Injection volume: 10 μl
Tray temp: Room temperature
Instrument: Waters Alliance 2695
Detector: Waters 996 Photo Diode Array
Wavelength: 238 nm
Eluens: A: 33% CH₃CN, 0.025% CF₃CO₂H in milliQ water
   B: 80% CH₃CN in milliQ water
   C: milliQ water

Two different gradients were used:

Gradient 1

| | Eluens (%) | | |
|---|---|---|---|
| Time (min) | A | B | C |
| 0.0-8.0 | 100 | 0 | 0 |
| 8.0-8.1 | 100→0 | 0→100 | 0 |
| 8.1-12 | 0 | 100 | 0 |
| 12.0-13.0 | 0→100 | 100→0 | 0 |
| 13.0-14.0 | 100 | 0 | 0 |

| Retention times (gradient 1): | |
|---|---|
| Hydrolyzed compactin | 10.4 min |
| Compactin | 10.9 min |
| ML-236A | 2.6 min |

Gradient 2

| | Eluens (%) | | |
|---|---|---|---|
| Time (min) | A | B | C |
| 0.0-5.0 | 50 | 0 | 50 |
| 5.0-5.1 | 50→100 | 0 | 50→0 |
| 5.1-9.0 | 100 | 0 | 0 |
| 9.0-9.1 | 100→0 | 0→100 | 0 |
| 9.1-13.0 | 0 | 100 | 0 |
| 13.0-13.1 | 0→50 | 100→0 | 0→50 |
| 13.1-15.0 | 50 | 0 | 50 |

| Retention times (gradient 2): | |
|---|---|
| Hydrolyzed compactin | 11.5 min |
| Compactin | 11.8 min |
| ML-236A | 8.6 min |

The wild type Penicillium citrinum strains barely produce any statins, while the Penicillium chrysogenum transformants produce significant amounts (Table 3). These data confirm the high potential of using derivatives of Penicillium chrysogenum industrial production strains as host cells for the production compactin.

TABLE 3
Statin levels (compactin and ML-236A) produced by different strains.

| | Compactin | ML-236A |
|---|---|---|
| Strain | (mg/L) | (mg/L) |
| Penicillium citrinum NRRL8082 | <0.5 | 0 |
| Penicillium citrinum NRRL8082 | <0.5 | 0 |
| Penicillium citrinum NRRL8082 | 0.9 | 0 |
| Penicillium citrinum NRRL8082 | <0.5 | 0 |
| Penicillium chrysogenum β-lactam free isolates | 0 | 0 |
| Penicillium chrysogenum β-lactam free isolates | 0 | 0 |
| Compactin cluster transformant #1 | 10 | 465 |
| Compactin cluster transformant #2 | 7 | 420 |

### Example 3

### Transformation of the Compactin Cluster Transformants #1 and #2 (Table 3) with a P450 Hydroxylase Enzyme: Fermentation of Pravastatin

A gene coding for a P450 compactin hydroxylase enzyme was produced synthetically (SEQ ID NO 19). The DNA was restricted with restriction enzymes NcoI and EcoRV; the resulting 1.2 kb fragment (partial digestion was performed due to internal EcoRV site, but the shorter 1 kb fragment was discarded) was cloned into the fungal expression vector pAN8-I (Punt & van den Hondel, 1993, Meth. Enzymology 216:447-457) digested NcoI and SmaI. The obtained integration construct pANP450, checked by restriction analysis, contains the P450 compactin hydroxylase gene downstream the Aspergillus nidulans gpdA promoter. The linearized pANP450 plasmid was co-transformed to the Penicillium chrysogenum compactin cluster transformants #1 and #2 with an amdS expression cassette encoding for a protein that enables the utilization of acetamide as sole nitrogen source. The amdS expression cassette was obtained by digesting pHELY-A1 (WO 04/106347) with NotI and isolating the 3.1 kb PgpdA-AnamdS expression cassette. Selection of transformants was done on mineral medium agar plates with 0.1% acetamide and 1 M saccharose. Colonies appearing on these protoplast regeneration plates were re-streaked on fresh acetamide agar plates without saccharose and grown until sporulation. The integration of the P450 expression cassette in amdS+ strain was verified by PCR, using oligonucleotides SEQ ID NO 20 and 21. Shake flasks experiments of transformants harboring the P450 expression cassette were carried out according to the method disclosed in Example 2. In the samples with P450 compactin hydroxylase containing Penicillium chrysogenum strains, a new peak with a retention time of 4.0 min eluted during the HPLC method 2, see Example 2, which could be assigned to Pravastatin. The formation of Pravastatin was further verified by analytical methods such as NMR and LC-MS/MS. For results see Table 4.

TABLE 4

| | Pravastatin | Compactin | ML-236A |
|---|---|---|---|
| Strain | (mg/L) | (mg/L) | (mg/L) |
| Compactin cluster transformant #1 | 0 | 9 | 467 |
| Compactin cluster transformant #2 | 0 | 7 | 420 |
| Compactin cluster transformants #1 | 242 | 50 | 110 |
| P450 clone 1 | | | |
| Compactin cluster transformants #1 | 150 | 22 | 80 |
| P450 clone 2 | | | |
| Compactin cluster transformants #2 | 211 | 10 | 130 |
| P450 clone 1 | | | |
| Compactin cluster transformants #2 | 87 | 45 | 73 |
| P450 clone 2 | | | |

### Example 4

### Transformation of the Compactin Cluster Genes from Penicillium citrinum into Lacking the Transcription Regulator mlcR into Penicillium chrysogenum

Penicillium chrysogenum β-lactam minus strains were transformed with the compactin biosynthetic genes lacking the regulator mlcR. This was achieved by digesting the plasmid pDONRP2-P3-6 kb right fragment compactin cluster (see Example 1) with BsaAl. By doing so, the ˜2 kb fragment harboring the mlcR gene was cut and removed by agarose gel electrophoresis. The remaining 6.6 kb plasmid fragment containing the gene mlcH was used in the co-transformation with the other two compactin cluster plasmids harboring the 18 kb fragment and the 14 kb fragment (see Example 1). As a selection marker, the ble expression cassette was co-transformed. The transformation of the β-lactam minus Penicillium chrysogenum strain was carried out in exact analogy to the experiment described in Example 2. Analysis of positive transformants and the performance of shake flask experiments (Penicillium chrysogenum strains with integrated ble expression cassettes as well as all three compactin gene cluster fragments) were analyzed as described in Examples 1 and 2. The results of the shake flask experiments revealed a significant decrease of the statin titers if the transcription regulator mlcR is missing. Some transformants show very low statin titers, while others do not give any detectable statin production any more. See Table 5.

TABLE 5

| Strain | Compactin (mg/L) | ML-236A (mg/L) |
|---|---|---|
| Compactin cluster transformant #1 | 9 | 467 |
| Compactin cluster transformant #2 | 7 | 420 |
| Transformant-mlcR #1 | 5 | 31 |
| Transformant-mlcR #2 | 0 | 0 |
| Transformant-mlcR #3 | 2 | 10 |
| Transformant-mlcR #4 | 12 | 14 |

### Example 5

### Transformation of the mlcR Minus Strains Obtained in Example 4 with mlcR and lovE Expression Cassettes

For the transformation, two new expression cassettes were constructed: a mlcR expression cassette and a lovE expression cassette. Both cassettes use identical promoter/terminator regions in order to facilitate comparisons between the transcription regulators mlcR and lovE. The two genes of mlcR (SEQ ID NO 23) and lovE (SEQ ID NO 22) were ordered synthetically. Both polynucleotides were digested with NcoI and EcoRV. The resulting 1.52 kb fragment (for lovE) and 1.39 kb fragment (for mlcR) was cloned into the vector fungal expression vector pAN8-I (Punt & van den Hondel, 1993, Meth. Enzymology 216:447-457) digested NcoI and SmaI. The obtained integration constructs pANlovE and pANmlcR, respectively, checked by restriction analysis, contain the transcription regulator gene downstream the Aspergillus nidulans gpdA promoter. Subsequently, the strains obtained in Example 4 (which contain all compactin biosynthetic genes except of the transcription regulator mlcR) were in a first step made amdS marker free by counter selection on fluoroacetamide (Hynes, M J and Pateman, J A (1970), Mol. Gen. Genetics, 108, 107-116). Only strains which do not harbor functional amdS expression cassettes can grow on fluoroacetamide, which otherwise is toxic. AmdS negative strains were furthermore transformed with either of the mlcR or lovE expression cassette harboring linearized plasmids (a suitable unique restriction site of the vector backbone was chosen), the P450 compactin hydroxylase expression cassette pANP450 (also here a linearized plasmid containing the expression cassette was used) and co-transformation of a amdS expression cassette. The amdS expression cassette was obtained by digesting pHELY-A1 (described in WO 2004/106347) with NotI and isolating the 3.1 kb PgpdA-AnamdS expression cassette. Selection of transformants was done on mineral medium agar plates with 0.1% acetamide and 1 M saccharose. Colonies appearing on these protoplast regeneration plates were re-streaked on fresh acetamide agar plates without the saccharose and grown until sporulation. Positive colonies (i.e. the integration of the mlcR or lovE expression cassette, and/or the p450 compactin hydroxylase) were identified by colony PCR (SEQ ID NO 20 and 21 for the P450 compactin hydroxylase gene, SEQ ID NO 26 and 27 for the lovE gene and SEQ ID NO 24 and 25 for the mlcR gene). Shake flasks were carried out according to the method disclosed in example 2. In the samples with lovE or mlcR containing Penicillium chrysogenum strains, a peak eluted during the HPLC method which could be assigned to pravastatin (retention time 4.0 minutes with HPLC method 2, see example 2). For results, see Table 6. The results clearly show 1) that mlcR as well as lovE are able to induce the formation of statins (pravastatin, compactin and ML236A) and 2) that lovE is a stronger inducer of statin production (especially pravastatin production) than mlcR.

TABLE 6
Shake flasks results of selected transformants

| | Pravastatin | Compactin | ML-236A |
|---|---|---|---|
| Strain | (mg/L) | (mg/L) | (mg/L) |
| Transformant-mIcR #1 (strain A) | 0 | 5 | 31 |
| Transformant-mIcR #2 (strain B) | 0 | 0 | 0 |
| Strain A + lovE | 0 | 212 | 681 |
| Strain A + mlcR | 0 | 10 | 452 |
| Strain A + lovE + P450 compactin | 952 | 50 | 137 |
| hydroxylase | | | |
| Strain A + mlcR + P450 compactin | 331 | 46 | 401 |
| hydroxylase | | | |

1. Fungal strain comprising a heterologous statin biosynthetic gene transcription activator which is the gene lovE or a polynucleotide that is at least 75% homologous to lovE, characterized in that said fungal strain is not Aspergillus terreus.

2. Fungal strain comprising the polypeptide LovE or a polypeptide that is at least 50% homologous to LovE, characterized in that said fungal strain is not Aspergillus terreus.

3. Fungal strain according to claim 1 which is Penicillium chrysogenum.

4. Fungal strain according to claim 1 which produces a statin chosen from the list consisting of compactin, lovastatin, monacolin J, pravastatin, simvastatin and wuxistatin.

5. Method for the production of a statin comprising fermenting a fungal strain according to claim 1.

6. Method according to claim 5 wherein said statin is compactin further comprising introduction into said fungal strain one or more of the genes mlcA, mlcB, mlcC, mlcD, mlcE, mlcF, mlcG and mlcH, or homologous genes with similar activity.

7. Method according to claim 5 wherein said statin is pravastatin further comprising introduction into said fungal strain one or more of the genes mlcA, mlcB, mlcC, mlcD, mlcE, mlcF, mlcG, mlcH, and a gene encoding a P450 compactin hydroxylase enzyme, or homologous genes with similar activity.

8. Method according to claim 5 wherein said statin is monacolin J further comprising introduction into said fungal strain one or more of the genes lovB, mlcC, mlcD, mlcE, mlcF, mlcG and mlcH, or homologous genes with similar activity.

9. Method according to claim 5 wherein said statin is lovastatin further comprising introduction into said fungal strain one or more of the genes lovB, mlcB, mlcC, mlcD, mlcE, mlcF, mlcG, and mlcH, or homologous genes with similar activity.

10. Method according to claim 5 wherein said statin is simvastatin further comprising introduction into said fungal strain one or more of the genes lovB, mlcC, mlcD, mlcE, mlcF, mlcG and mlcH (or homologous genes with similar activity) and wherein 2,2-dimethylbutyrate or a 2,2-dimethylbutyrate precursor is added to a culture of said fungal strain.

11. Use of a strain according to claim 1 for the preparation of a statin.

12. Use according to claim 11 wherein said statin is produced via enzymes encoded by heterologous genes.
